(19) United States
(12) Patent Application Publication
(10) Pub. No.: US 2024/0000337 A1
(43) Pub. Date: Jan. 4, 2024

(54) Wearable Device Including Optical Sensor Circuitry

(71) Applicant: Apple Inc., Cupertino, CA (US)

(72) Inventors: Tong Chen, Fremont, CA (US); Ahmet Fatih Cihan, San Jose, CA (US); Adrian Z. Harb, San Jose, CA (US); Nicholas P. Allec, Santa Cruz, CA (US); Joel S. Armstrong-Muntner, San Carlos, CA (US); James E. Stark, Mountain View, CA (US)

(21) Appl. No.: 18/202,167

(22) Filed: May 25, 2023

Related U.S. Application Data
(60) Provisional application No. 63/356,955, filed on Jun. 29, 2022.

(51) Int. Cl. A61B 5/08; A61B 5/00

(52) U.S. Cl. CPC A61B 5/08; A61B 5/0059; A61B 5/6803; A61B 5/7445

(57) ABSTRACT

A head mounted device includes a housing and a set of one or more SMI sensors. The set of one or more SMI sensors are disposed in the housing. The set of one or more SMI sensors are configured to emit electromagnetic radiation toward an anatomical structure adjacent a nasal passageway of a user and generate one or more SMI signals including information about movement of the anatomical structure.

### CROSS-REFERENCE TO RELATED APPLICATION

This application is a nonprovisional and claims the benefit under 35 U.S.C. § 119(e) of U.S. Provisional Patent Application No. 63/356,955, filed Jun. 29, 2022, the contents of which are incorporated herein by reference as if fully disclosed herein.

### TECHNICAL FIELD

The described embodiments generally relate to wearable devices, and in particular to wearable devices that include interferometric sensors, such as self-mixing interferometry (SMI) sensors, and to wearable devices that use such sensors to sense various physical phenomena.

### BACKGROUND

Wearable devices such as smart watches, smart eyewear, virtual and/or augmented reality headsets, and the like, may include various sensors, which may sense physical phenomena such as movement, environmental conditions, and biometric data about a user. The data from sensors in a wearable device may be used to provide valuable information to a user, such as information about the activity and/or health of the user. Additional sensors in wearable devices may provide more robust information to a user and/or control or unlock additional applications of the wearable device. Given the wide range of applications for sensors in wearable devices, any new development in the configuration or operation of the sensors therein can be useful. New developments that may be particularly useful are developments that provide additional sensing capability while maintaining a small form factor.

### SUMMARY

Embodiments of the systems, devices, methods, and apparatus described in the present disclosure are directed to the configuration and operation of sensors for wearable devices. The sensors may include interferometric sensors such as SMI sensors. The sensors may be positioned and oriented within the wearable device to sense physical phenomena related to one or more anatomical structures of a user, such as movement of an anatomical structure adjacent to a nasal passageway of the user. Processing circuitry of the wearable device may determine respiration information about the user based on SMI signals from the SMI sensors. The wearable device may be a head-mounted device such as eyewear, a virtual and/or augmented reality headset, a nose clip, or a face mask, and the SMI sensors may be positioned and oriented over the nose of the user. The wearable device may be operated to detect when it is appropriate to emit electromagnetic radiation (e.g., when electromagnetic radiation will not be directed towards the eyes of the user) and enabling or disabling electromagnetic radiation from the SMI sensors when it is appropriate to do so.

In a first aspect, a head-mounted device may include a housing and a set of one or more SMI sensors disposed in the housing. Each of the set of one or more SMI sensors may be configured to emit electromagnetic radiation toward an anatomical structure adjacent a nasal passageway of a user and generate one or more SMI signals including information about movement of the anatomical structure. Processing circuitry may be communicably coupled to the set of one or more SMI sensors and configured to determine respiration information about the user based on the one or more SMI signals. The respiration information may include one or more of respiration rate, respiration quality, information about nasal congestion, information about snoring, airflow velocity, and airflow volume. The processing circuitry may further be configured to detect a facial movement of the user based on the one or more SMI signals. A display may be communicably coupled to the processing circuitry. The processing circuitry may change a user interface shown on the display based on the detection of the facial movement from the user.

In another aspect, a wearable device may include a housing, a set of one or more SMI sensors disposed in the housing, and processing circuitry communicably coupled to the set of one or more SMI sensors. Each of the set of one or more SMI sensors may be configured to emit electromagnetic radiation towards an anatomical structure of a user and generate one or more SMI signals including information about the anatomical structure. The processing circuitry may be configured to determine if it is appropriate to emit electromagnetic radiation from the set of one or more SMI sensors, enable the emission of electromagnetic radiation from the set of one or more SMI sensors when it is determined to be appropriate to do so, and disable the emission of electromagnetic radiation from the set of one or more SMI sensors when it is not determined appropriate to do so. The processing circuitry may determine if it is appropriate to emit electromagnetic radiation from the set of one or more SMI sensors based on the one or more SMI signals, and/or based on a proximity signal from a proximity sensor disposed in the housing.

In another aspect, a method of operating a wearable device may include generating, from a set of one or more SMI sensors of the wearable device, one or more SMI signals including information about movement of tissue near a respiratory pathway of a user, and determining, by a processing system of the wearable device, respiration information about the user based on the one or more SMI signals. The respiration information may include one or more of respiration rate, respiration quality, information about nasal congestion, information about snoring, airflow velocity, and airflow volume. The tissue may be bone or soft tissue. The respiratory pathway may be a nasal passageway.

In addition to the exemplary aspects and embodiments described above, further aspects and embodiments will become apparent by reference to the drawings and by study of the following description.

### BRIEF DESCRIPTION OF THE DRAWINGS

Reference will now be made to representative embodiments illustrated in the accompanying figures. It should be understood that the following descriptions are not intended to limit this disclosure to one included embodiment. To the contrary, the disclosure provided herein is intended to cover alternatives, modifications, and equivalents as may be included within the spirit and scope of the described embodiments, and as defined by the appended claims.

FIG. 1 shows an example electrical block diagram of a wearable device, such as described herein.

FIGS. 2A and 2B show an anatomical views of a nose, such as described herein.

FIG. 3 shows an exemplary wearable device being worn by a user, such as described herein.

FIG. 4 shows an exemplary wearable device being worn by a user, such as described herein.

FIGS. 5A and 5B show an exemplary wearable device being worn by a user, such as described herein.

FIG. 6 is a flow diagram illustrating a method of operating a wearable device, such as described herein.

FIG. 7 is a flow diagram illustrating a method of operating a wearable device, such as described herein.

FIG. 8 is an example electrical block diagram of a wearable device, such as described herein.

The use of the same or similar reference numerals in different figures indicates similar, related, or identical items.

The use of cross-hatching or shading in the accompanying figures is generally provided to clarify the boundaries between adjacent elements and also to facilitate legibility of the figures. Accordingly, neither the presence nor the absence of cross-hatching or shading conveys or indicates any preference or requirement for particular materials, material properties, element proportions, element dimensions, commonalities of similarly illustrated elements, or any other characteristic, attribute, or property for any element illustrated in the accompanying figures.

Additionally, it should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Coherent optical sensing, including Doppler velocimetry and heterodyning, can be used to measure physical phenomena including presence, distance, velocity, size, surface properties, and particle count. Interferometric sensors such as SMI sensors may be used to perform coherent optical sensing. An SMI sensor is defined herein as a sensor that is configured to generate and emit light from a resonant cavity of a semiconductor light source, receive a reflection or backscatter of the light (e.g., light reflected or backscattered from an object) back into the resonant cavity, coherently or partially coherently self-mix the generated and reflected/backscattered light within the resonant cavity, and produce an output indicative of the self-mixing (i.e., an SMI signal). The generated, emitted, and received light may be coherent or partially coherent, but a semiconductor light source capable of producing such coherent or partially coherent light may be referred to herein as a coherent light source. The generated, emitted, and received light may include, for example, visible or invisible light (e.g., green light, infrared (IR) light, or ultraviolet (UV) light). The output of an SMI sensor (i.e., the SMI signal) may include a photocurrent produced by a photodetector (e.g., a photodiode). Alternatively or additionally, the output of an SMI sensor may include a measurement of a current or junction voltage of the SMI sensor's semiconductor light source.

Generally, an SMI sensor may include a light source and, optionally, a photodetector. The light source and photodetector may be integrated into a monolithic structure. Examples of semiconductor light sources that can be integrated with a photodetector include vertical cavity surface-emitting lasers (VCSELs), edge-emitting lasers (EELs), horizontal cavity surface-emitting lasers (HCSELs), vertical external-cavity surface-emitting lasers (VECSELs), quantum-dot lasers (QDLs), quantum cascade lasers (QCLs), and light-emitting diodes (LEDs) (e.g., organic LEDs (OLEDs), resonant-cavity LEDs (RC-LEDs), micro LEDs (mLEDs), superluminescent LEDs (SLEDS), and edge-emitting (ELEDs). These light sources may also be referred to as coherent light sources. A semiconductor light source may be integrated with a photodetector in an intra-cavity, stacked, or adjacent photodetector configuration to provide an SMI sensor.

Generally, SMI sensors have a small footprint and are capable of measuring myriad physical phenomena. Accordingly, they are useful in wearable devices, which are generally limited in size. As discussed above, a portion of the functionality of many wearable devices is directed to the measurement of biometric data about a user, such as heart rate and respiration rate. The small footprint of SMI sensors may enable additional sensing opportunities by allowing sensors to be placed in previously impractical locations, while the high accuracy of SMI sensors may enable the determination of rich biometric data.

As described in various embodiments herein, SMI sensors may be used to determine biometric data such as movement, and in particular muscle, ligament, tendon, and/or skin movement, and respiratory information such as respiration rate, respiration quality, information about nasal congestion, information about snoring, airflow velocity, and breathing volume. Placing and orienting SMI sensors in a head-mounted device so that they emit electromagnetic radiation towards an anatomical structure adjacent to a nasal passageway of a user may allow for the accurate determination of respiration information based on movement of the anatomical structure. For example, placing and orienting SMI sensors over a portion of the nose of the user may allow a head-mounted device such as smart eyewear, a virtual and/or augmented reality headset, a smart face-mask, and/or a smart nose clip to determine respiration information about a user.

SMI sensors may additionally or alternatively be used to detect intentional or unintentional movement of the face and/or nose of the user. Detection of unintentional facial movements may provide data useful for the diagnosis or monitoring of a health condition. Detection of intentional movements may be used to control various aspects of a device, such as navigating a user interface thereof.

Nasal and/or eye tissue, for example, of users can have various sensitivities, such as allergies, abrasion sensitivities, sensor and/or energy exposure sensitivities, etc. Accordingly, in some aspects described herein SMI sensors may be operated to emit electromagnetic radiation for sensing only when it is determined to be appropriate. For example, SMI sensors may be operated to emit electromagnetic radiation when they are in contact with a user's skin or the electromagnetic radiation emitted therefrom is otherwise unlikely to be directed at or towards a user's eyes. Accordingly, wearable devices described herein may detect when it is appropriate to emit electromagnetic radiation from a particular SMI sensor or sensors and enable and disable the emission of electromagnetic radiation therefrom accordingly.

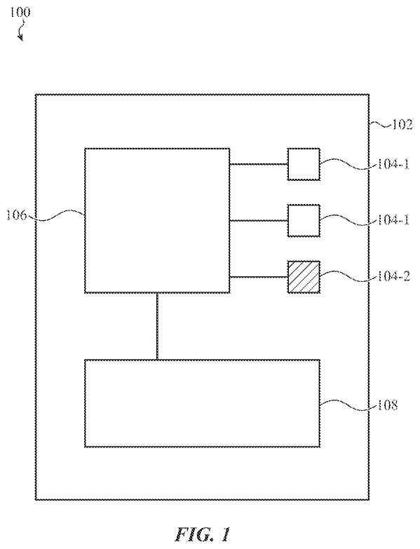

The foregoing and other embodiments are discussed below with reference to FIGS. 1-8. However, those skilled in the art will readily appreciate that the detailed description given herein with respect to these figures is for explanation only and should not be construed as limiting.

Directional terminology, such as “top”, “bottom”, “upper”, “lower”, “front”, “back”, “over”, “under”, “above”, “below”, “left”, or “right” is used with reference to the orientation of some of the components in some of the figures described below. Because components in various embodiments can be positioned in a number of different orientations, directional terminology is used for purposes of illustration only and is usually not limiting. The directional terminology is intended to be construed broadly, and therefore should not be interpreted to preclude components being oriented in different ways. Also, as used herein, the phrase “at least one of” preceding a series of items, with the term “and” or “or” to separate any of the items, modifies the list as a whole, rather than each member of the list. The phrase “at least one of” does not require selection of at least one of each item listed; rather, the phrase allows a meaning that includes at a minimum one of any of the items, and/or at a minimum one of any combination of the items, and/or at a minimum one of each of the items. By way of example, the phrases “at least one of A, B, and C” or “at least one of A, B, or C” each refer to only A, only B, or only C; any combination of A, B, and C; and/or one or more of each of A, B, and C. Similarly, it may be appreciated that an order of elements presented for a conjunctive or disjunctive list provided herein should not be construed as limiting the disclosure to only that order provided.

FIG. 1 shows an exemplary wearable device 100. The wearable device 100 includes a housing 102, a number of sensors 104 disposed in the housing 102, processing circuitry 106 communicably coupled to the sensors 104, and a display 108, which is also communicably coupled to the processing circuitry 106. The sensors 104 may include a number of SMI sensors 104-1 and a proximity sensor 104-2. While two SMI sensors 104-1 and one proximity sensor 104-2 are shown for purposes of illustration, the wearable device 100 may include any number of SMI sensors 104-1 and any number of proximity sensors 104-2. Further, the wearable device 100 may include any number of additional sensors, which are not shown. As discussed herein, the sensors 104 may be positioned and oriented in the housing 102 to emit electromagnetic radiation towards an anatomical structure adjacent a nasal passageway of the user. For example, the sensors 104 may be positioned and oriented to be over or otherwise near the nose of the user when the wearable device 100 is being worn. A display 108 may be positioned to be in front an eye of the user. In some aspects of the present disclosure, two displays 108 may be provided, one in front of each eye of the user. In another aspect, the wearable device 100 does not include a display, and the user may interact with the wearable device 100 using a non-visual user interface (e.g., voice control) or interact with the wearable device 100 via a device that is communicably coupled to the wearable device 100 (e.g., via a wired or wireless connection).

The SMI sensors 104-1 may be operated to emit electromagnetic radiation toward an anatomical structure of the user adjacent a nasal passageway. For example, the SMI sensors 104-1 may be positioned and oriented to emit electromagnetic radiation toward tissue adjacent, surrounding, or otherwise near the nasal passageway of the user. The tissue may be bone or soft tissue. For example, the SMI sensors 104-1 may be positioned and oriented to emit electromagnetic radiation towards a nasal bone of the user, an upper lateral cartilage of the nose of the user, a lower lateral cartilage of the nose the user, and/or the skin on and/or around the nose of the user. The SMI sensors 104-1 may be positioned and oriented to be directly against the skin of the user, or there may be an air gap present between the SMI sensors 104-1 and the skin of the user. The electromagnetic radiation emitted from the SMI sensors 104-1 may be configured to reflect and/or backscatter from the tissue of the user or penetrate the tissue of the user to a desired depth, passing through some tissue (e.g., skin) with minimal or low reflection and/or backscatter, while reflecting and/or backscattering off other tissue (e.g., cartilage or bone) to a greater degree. For example, certain characteristics of the electromagnetic radiation (e.g., wavelength) and/or a focal length of the SMI sensors 104-1 may be configured to measure movement of a desired anatomical structure. In some aspects, different ones of the SMI sensors 104-1 may be configured to emit electromagnetic radiation that reflects and/or backscatters primarily from different anatomical structures, either by the position and orientation of the SMI sensors 104-1 in the housing 102, or by the characteristics of the electromagnetic radiation emitted therefrom.

The electromagnetic radiation emitted from the SMI sensors 104-1 may be modulated or non-modulated. The modulation, or lack of modulation, of the electromagnetic radiation may allow for the detection of different physical phenomena. For example, a first modulation pattern of the electromagnetic radiation emitted from the SMI sensors 104-1 may be useful for detecting the proximity of an object to the SMI sensors 104-1, while a second modulation pattern of the electromagnetic radiation emitted from the SMI sensors 104-1 may be useful for detecting movement (e.g., velocity) of an object. In various aspects, the SMI sensors 104-1 may be operated such that the electromagnetic radiation emitted therefrom is modulated in the same or different ways in order to detect desired physical phenomena.

The electromagnetic radiation emitted from an SMI sensor 104-1 may be partially reflected and/or backscattered from a desired anatomical structure back towards the SMI sensor 104-1. The reflected and/or backscattered electromagnetic radiation may self-mix (or interfere) with the generated electromagnetic radiation. The self-mixing may be measured (e.g., by measuring the electromagnetic radiation with a photodetector or by measuring a current and/or junction voltage of a light source of the SMI sensor 104-1) to generate an SMI signal. By generating the electromagnetic radiation via specific drive patterns (e.g., via doppler and/or triangular drive patterns) and measuring the reflection and/or backscatter thereof, SMI signals may include information about movement of the desired anatomical structure.

The proximity sensor 104-2 may detect the proximity of the wearable device 100 to the user, which is indicated in a proximity signal provided to the processing circuitry 106. The proximity sensor 104-2 may be any suitable type of proximity sensor, such as, for example, an ultrasonic sensor, an infrared sensor, a capacitive sensor, or a resistive sensor. In general, it may be desirable for the proximity sensor 104-2 to be a type of proximity sensor that prevents the SMI sensor 104-1 from emitting electromagnetic radiation, as discussed herein.

As discussed above, the desired anatomical structure may be adjacent a nasal passageway of the user. For example, the desired anatomical structure may be the nasal bone, the upper lateral cartilage of the nose, the lower lateral cartilage of the nose, and/or the skin on and/or around the nose. The processing circuitry 106 may use the information about movement of the desired anatomical structure in the SMI signals to determine respiration information about the user. For example, the processing circuitry 106 may use the information about movement of the desired anatomical structure to determine respiration rate, respiration quality, information about nasal congestion (e.g., a degree of nasal congestion), information about snoring (e.g., the presence or absence of snoring, a severity of snoring), airflow velocity, and breathing volume. The processing circuitry 106 may determine respiration information from the SMI signals in any suitable manner, such as, for example, by providing the SMI signals to a machine learning model.

In addition to respiration information, the processing circuitry 106 may also use the SMI signals to determine voluntary or involuntary facial movements of the user. For example, the processing circuitry 106 may use the SMI signals to detect facial tics of a user, which may provide information for the diagnosis or monitoring of some health conditions. Additionally, the processing circuitry 106 may use the SMI signals to detect intentional facial movements, such as a movement of the nose. Detected intentional facial movements may be used, for example, as a user input to the wearable device 100. For example, intentional facial movements of the user, in addition to other types of user input, may be used to change or otherwise navigate a user interface shown on the display 108 of the wearable device 100. Notably, the display 108 may be omitted in certain aspects and intentional facial movements may be used as a user input to control or otherwise operate the wearable device 100 in any suitable manner.

While not shown, the wearable device 100 may include any number of user input elements such as buttons, microphones, speakers, or the like. The wearable device 100 may also include additional structural elements such as straps, bands, or other suitable elements for positioning, attaching, or securing the wearable device 100 to the user. The wearable device 100 may also include additional circuitry, such as additional sensors, communication circuitry (e.g., wired or wireless communication circuitry), or any other circuitry to facilitate the operation and functionality of the wearable device 100.

The wearable device 100 may be a head-mounted device. Accordingly, the housing 102 of the wearable device may be shaped and sized to be mounted to the head of a user. One or more straps or other mounting structures (not shown) may be used to affix the wearable device 100 to the head of the user. In various aspects discussed herein, the housing 102 may be sized and shaped to provide eyewear, a virtual and/or augmented reality headset, a face mask, and a nose clip. However, the form factor of the wearable device may be provided in any suitable shape and size without departing from the principles herein.

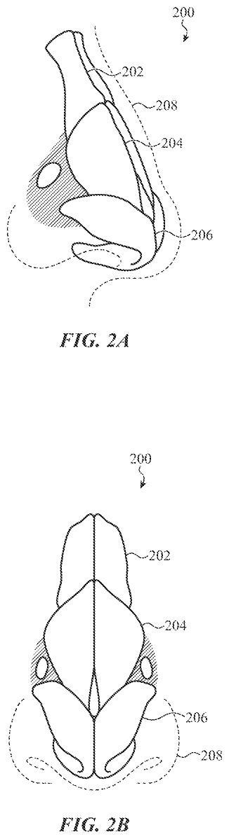

FIGS. 2A and 2B show anatomical views of a nose 200 of a user. In particular, FIG. 2A shows an anatomical view of a nose of a user along a sagittal plane, while FIG. 2B shows an anatomical view of a nose of a user along a frontal plane. The nose 200 includes a nasal bone 202, an upper lateral cartilage 204, and a lower lateral cartilage 206. The nasal bone 202, upper lateral cartilage 204, and lower lateral cartilage 206 are covered with skin 208. In various aspects of the present disclosure, SMI sensors such as those discussed herein may be positioned and oriented in a wearable device such that they emit electromagnetic radiation towards one or more of the nasal bone 202, the upper lateral cartilage 204, the lower lateral cartilage 206, and the skin 208 on or around the nose 200. Movement of any one of the nasal bone 202, the upper lateral cartilage 204, the lower lateral cartilage 206, and the skin 208 on or around the nose 200 may be indicative of various respiration information about the user such as respiration rate, respiration quality, information about nasal congestion (e.g., a degree of nasal congestion), information about snoring (e.g., the presence or absence of snoring, severity of snoring), airflow velocity, and breathing volume. As discussed herein, the electromagnetic radiation emitted from the SMI sensors may be configured (e.g., via wavelength, focal length, etc.) to primarily reflect and/or backscatter from a particular one of the aforementioned anatomical structures, or any other anatomical structure, and measured to generate SMI signals that are used to determine the respiration information. Further, the SMI signals may be used to detect voluntary and involuntary nose and/or facial movements.

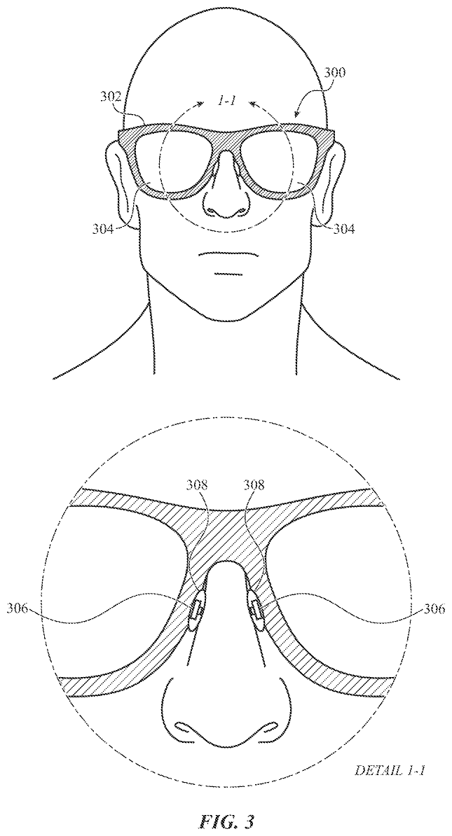

FIG. 3 shows a wearable device 300 being worn by a user according to an additional aspect of the present disclosure. The wearable device 300 shown in FIG. 3 is in the form factor of eyewear, and thus may include a frame 302, a pair of lenses 304, and a number of sensors 306, which may be positioned and oriented in nosepieces 308 coupled to the frame 302 such that they are over or near the nose of the user. An enlarged view of the nosepieces 308 including the sensors 306 is shown in FIG. 3. The sensors 306 may be positioned and oriented so that they emit electromagnetic radiation towards an anatomical structure adjacent a nasal passageway of the user. The sensors 306 may be positioned and oriented in the nosepieces 308 such that they are in direct contact with the skin of the user or such that there is an air gap between the sensors 306 and the skin of the user. The sensors 306 may be SMI sensors or include at least one SMI sensor along with one or more other types of sensors, such as a proximity sensor. The sensors 306 may be operated as discussed herein to detect movement of an anatomical structure of a user, determine respiration information about the user, detect intentional and/or unintentional facial movements of the user, and operate appropriately to avoid irritating a user. While the nosepieces 308 are shown as separate pieces coupled to the frame 302, in some aspects the nosepieces 308 may be molded into or otherwise integrated with the frame 302. While not shown, the wearable device 300 may include a display, which may be projected or otherwise provided on one or both of the lenses 304. In some aspects, the wearable device 300 may not include a display. Further, the wearable device 300 may include processing circuitry to operate the sensors 306 as discussed herein, additional circuitry, additional user input elements such as buttons, microphones, speakers, and cameras, and/or additional structural elements. In general, FIG. 3 is meant to illustrate an exemplary form factor of a wearable device 300 as discussed herein, as well as the placement of sensors 306 in the exemplary form factor.

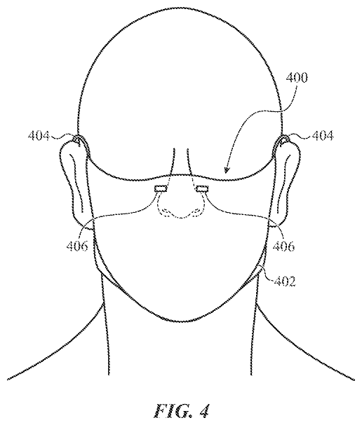

FIG. 4 shows a wearable device 400 being worn by a user according to an additional aspect of the present disclosure. The wearable device 400 shown in FIG. 4 is in the form factor of a face mask, and thus may include a cover 402, a number of straps 404 coupled to the cover 402 and configured to attach the cover 402 over the nose and/or mouth of the user, and a number of sensors 406 disposed in the cover 402. The sensors 406 may be positioned and oriented to be over or near the nose of the user. In particular, the sensors 406 may be positioned and oriented to emit electromagnetic radiation towards an anatomical structure adjacent a nasal passageway of the user. In various aspects, the sensors 406 may be in direct contact with the skin of the user or there may be an air gap between the sensors 406 and the skin of the user. The sensors 406 may be SMI sensors or include at least one SMI sensor along with one or more other types of sensors, such as a proximity sensor. The sensors 406 may be operated as discussed herein to detect movement of an anatomical structure of a user, determine respiration information about the user, detect intentional and/or unintentional facial movements of the user, and operate appropriately to avoid irritating a user. While not shown, the wearable device 400 may include additional components such as a display, processing circuitry to operate the sensors 406 as discussed herein, additional circuitry, additional user input elements such as buttons, microphones, speakers, and cameras, and/or additional structural elements. In general, FIG. 4 is meant to illustrate an exemplary form factor of a wearable device 400 as discussed herein, as well as the placement of sensors 406 in the exemplary form factor.

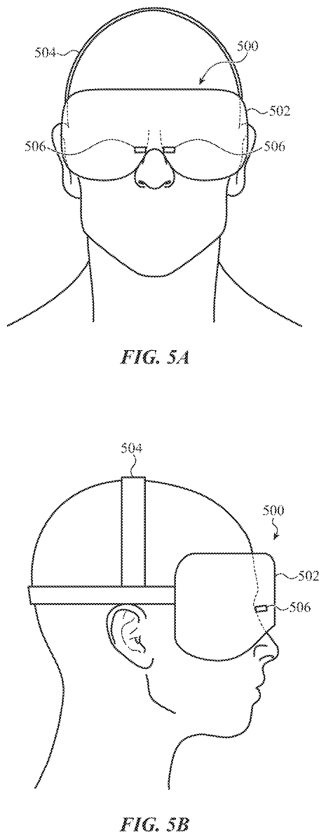

FIGS. 5A and 5B show a wearable device 500 being worn by a user according to an additional embodiment of the present disclosure. In particular, FIG. 5A shows a front view and FIG. 5B shows a side view of the wearable device 500 being worn by the user. The wearable device 500 shown in FIGS. 5A and 5B is in the form factor of a virtual and/or augmented reality headset, and thus may include a housing 502, a strap 504 for attaching the housing 502 to the head of the user, and a number of sensors 506 disposed in the housing 502. The sensors 506 may be positioned and oriented to be over or near the nose of the user. In particular, the sensors 506 may be positioned and oriented to emit electromagnetic radiation towards an anatomical structure adjacent a nasal passageway of the user. In various aspects, the sensors 506 may be in direct contact with the skin of the user or there may be an air gap between the sensors 506 and the skin of the user. The sensors 506 may be SMI sensors or include at least one SMI sensor along with one or more other types of sensors, such as a proximity sensor. The sensors 506 may be operated as discussed herein to detect movement of an anatomical structure of a user, determine respiration information about the user, detect intentional and/or unintentional facial movements of the user, and/or operate appropriately to avoid irritating a user. While not shown, the wearable device 500 may include additional components such as displays, processing circuitry to operate the displays and sensors 506 as discussed herein, additional circuitry, additional user input elements such as buttons, microphones, speakers, and cameras, and/or additional structural elements. In general, FIGS. 5A and 5B are meant to illustrate an exemplary form factor of a wearable device 500 as discussed herein, as well as the placement of sensors 506 in the exemplary form factor.

While FIGS. 3-5B illustrate various exemplary form factors of a wearable device, they are not meant to be exhaustive. The present disclosure contemplates any form factor for a wearable device capable of positioning SMI sensors as discussed herein, including swimming goggles, safety eyewear, or any other suitable form factor.

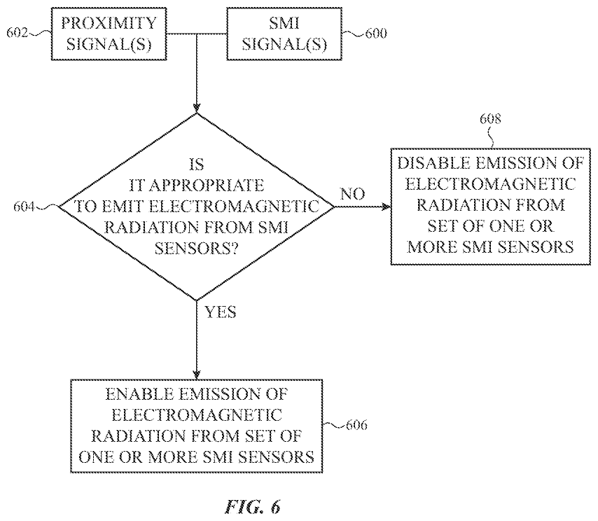

As discussed herein, SMI sensors may be placed over or near the nose of a user to determine valuable information such as respiration information as well as voluntary or involuntary nose and/or facial movements. In some instances, some users may be especially sensitive to electromagnetic radiation, and thus placing SMI sensors in close proximity to the eyes of the user may require additional considerations. Accordingly, FIG. 6 is a flow diagram illustrating a method of operating a wearable device according to one aspect of the present disclosure. One or more SMI signals are received from one or more SMI sensors (step 600). Additionally or alternatively, one or more proximity signals are received from one or more proximity sensors (step 602). The one or more SMI signals and/or the one more proximity signals are used by processing circuitry of the wearable device to determine if it is appropriate to emit electromagnetic radiation from the one or more SMI sensors (step 604). Determining if it is appropriate to emit electromagnetic radiation from the one or more SMI sensors may include determining if the wearable device is being worn by the user, or is being properly worn by the user (e.g., the SMI sensors and/or proximity sensors are directly against the skin of the user). Such a determination may be accomplished in any suitable manner, including comparing the SMI signals and/or proximity signals to a threshold value, making calculations based on the SMI signals and/or proximity signals, providing the SMI signals and/or proximity signals to a machine learning model, etc. If it is appropriate to emit electromagnetic radiation from the one or more SMI sensors, the processing circuitry may enable the emission of electromagnetic radiation from the one or more SMI sensors (step 606). Alternatively, if it is not appropriate to emit electromagnetic radiation from the one or more SMI sensors or there is not sufficient information from the proximity sensor, the processing circuitry may disable the emission of electromagnetic radiation from the one or more SMI sensors (step 608).

The foregoing process may be repeated at a predetermined interval, or initiated in response to a detected event such as significant movement of the wearable device, which may be detected by one or more additional sensors such as an accelerometer. Operating the SMI sensors of a wearable device in this manner may improve the safety profile, battery life, and/or efficacy of the device. The principles of operation described with respect to FIG. 6 may be used in any of the wearable devices described herein.

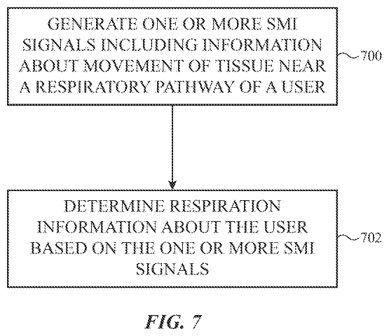

The wearable devices discussed herein may determine respiration information about a user based on SMI sensors positioned on or near the nose of the user. However, the present disclosure contemplates the broader use of information about movement of tissue near a respiratory pathway of a user to determine respiration information about the user. To illustrate these principles, FIG. 7 is a flow diagram describing a method for operating a wearable device to obtain respiration information about a user according to one aspect of the present disclosure. First, one or more SMI signals are generated, where the one or more SMI signals include information about the movement of tissue near a respiratory pathway of a user (step 700). The movement of the tissue may be a vibration of the tissue. The tissue may be soft tissue such as skin, cartilage, muscle, tendon, or ligament, or hard tissue such as bone. The one or more SMI signals may be generated from one or more SMI sensors in the wearable device. The one or more SMI sensors may be positioned and oriented to be over or near the respiratory pathway of the user. The respiratory pathway may be a nasal passageway of the user.

Next, respiration information about the user is determined based on the one or more SMI signals (step 702). The respiration information may be determined, for example, by providing the one or more SMI signals to a machine learning model. In general, any suitable calculation, transformation, or the like may be performed to determine the respiration information from the one or more SMI signals. The respiration information may include one or more of respiration rate, respiration quality, information about nasal congestion (i.e., degree of nasal congestion), information about snoring (e.g., presence or absence of snoring, severity of snoring), airflow velocity, and breathing volume. The respiration information may be useful to the user for the diagnosis or monitoring of some health conditions. In some aspects, the respiration information may be displayed graphically for the user. The wearable device may use the respiration information to notify the user of certain events, such as when the user is experiencing a certain level of nasal congestion (which may be indicative of seasonal allergies and/or illness), when the user is breathing through the mouth rather than the nose, etc. Further, visualizations of the user's breathing may be generated and shown to the user, which may aid in activities such as guided breathing instruction or biofeedback. If it is detected that a user stops breathing, emergency services can be contacted to provide medical aid, in some cases automatically. The principles of operation described with respect to FIG. 7 may be used in any of the wearable devices described herein.

In addition to determining respiration information, data from SMI sensors positioned and oriented to be over or near a respiratory pathway may be used along with complimentary data streams from other sensors to obtain or discern additional information about a user. The other sensors may be located in the wearable device itself, in a different wearable device worn by the user, in a wearable device worn by another user, or in a non-wearable device. For example, data from a wrist-worn wearable device worn by the user may be combined with data from SMI sensors in a wearable device as described herein to obtain additional information about the user. Further, data from a non-wearable device, such as a device in the environment around a user may be combined with data from SMI sensors in a wearable device as described herein to obtain additional information about a user. Data from a wearable device worn by a different user may also be combined with data from SMI sensors in a wearable device as described herein to enable additional functionality (e.g., improved gaming experiences between users). In one example, blood oxygen saturation information from a blood oxygen saturation sensor in a wearable device worn by the user may be combined with respiration information obtained as discussed herein. The blood oxygen saturation information may enrich the respiration information to enable discernment of respiration events such as a user holding their breath versus a user choking or drowning. Gaze tracking information may be combined with respiration information to determine information about a user such as attentiveness and focus on a task (e.g., student or driver focus), which may enable a user to be notified to take a break as attentiveness wanes. Respiration information, alone or combined with other information about a user, may enable a wearable device to provide breathing queues (e.g., when to breathe in, when to hold breath, when to breathe out, and breathing pacing), either for general health or related to a particular task such as for improving performance in sporting or other activities (e.g., swimming, diving, golf, tennis, archery, and baseball).

Respiration information, along with other complimentary information, may also be used to track a user's breathing or health trends over time. Such information may be indicative of training capacity and whether a particular training regimen is effective for a user. Respiration information, along with other complimentary information, may also provide an unobtrusive way to monitor an emotional response of a user, for example, by detecting gasping, laughter, crying, sobbing, speech and speech emphasis, etc. Monitoring emotional response via respiration information may be less obtrusive than directly monitoring audio. Emotional response information may in turn be useful in determining a user's response to various environmental stimuli or medications, which a user may wish to track over time.

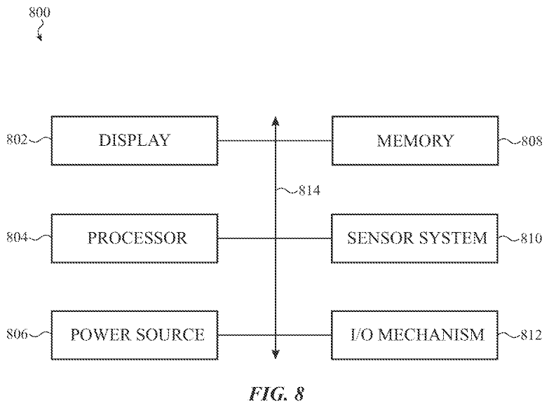

FIG. 8 shows a sample electrical block diagram of a wearable device 800, which may be implemented as any of the devices described with respect to FIGS. 1 and 3-5B. The wearable device 800 may include an electronic display 802 (e.g., a light-emitting display), a processor 804 (also referred to herein as processing circuitry), a power source 806, a memory 808, or storage device, a sensor system 810, an input/output (I/O) mechanism 812 (e.g., an input/output device, input/output port, or haptic input/output interface). The processor 804 may control some or all of the operations of the wearable device 800. The processor 804 may communicate, either directly or indirectly, with some or all of the other components of the wearable device 800. For example, a system bus or other communication mechanism 814 can provide communication between the electronic display 802, the processor 804, the power source 806, the memory 808, the sensor system 810, and the I/O mechanism 812.

The processor may be implemented as any electronic device capable of processing, receiving, or transmitting data or instructions, whether such data or instructions is in the form of software or firmware or otherwise encoded. For example, the processor 804 may include a microprocessor, central processing unit (CPU), an application-specific integrated circuit (ASIC), a digital signal processor (DSP), a controller, or a combination of such devices. As described herein, the term “processor” or “processing circuitry” is meant to encompass a single processing unit, multiple processors, multiple processing units, or other suitably configured computing element or elements. In some embodiments, the processor 804 may provide part or all of the processing systems, processing circuitry, or processors described with reference to any of FIGS. 1 and 3-5B.

It should be noted that the components of the wearable device 800 can be controlled by multiple processors. For example, select components of the wearable device 800 (e.g., the sensor system 810) may be controlled by a first processor and other components of the wearable device 800 (e.g., the electronic display 802) may be controlled by a second processor, where the first and second processors may or may not be in communication with each other.

The power source 806 can be implemented with any device capable of providing energy to the wearable device 800. For example, the power source 806 may include one or more batteries or rechargeable batteries. Additionally or alternatively, the power source 806 may include a power connector or power cord that connects the wearable device 800 to another power source, such as a wall outlet.

The memory 808 may store electronic data that can be used by the wearable device 800. For example, the memory 808 may store electrical data or content such as, for example, audio and video files, documents and applications, device settings and user preferences, timing signals, control signals, and data structures and databases. The memory 808 may include any type of memory. By way of example only, the memory 808 may include random access memory (RAM), read-only memory (ROM), flash memory, removeable memory, other types of storage elements, or combinations of such memory types.

The wearable device 800 may also include one or more sensor systems 810 positioned almost anywhere on the wearable device 800. For example, the sensor system 810 may include any and all of the sensors discussed herein with respect to FIGS. 1 and 3-5B. The sensor system 810 may be configured to sense one or more types of parameters, such as but not limited to: vibration, light, touch, force, heat, movement, relative motion, biometric data (e.g., biological parameters) of a user, air quality, proximity, position, or connectedness. By way of example, the sensor system 810 may include one or more SMI sensors as discussed herein with respect to FIGS. 1 and 3-5B, a heat sensor, a position sensor, a light or optical sensor, an accelerometer, a pressure transducer, a gyroscope, a magnetometer, a health monitoring sensor, and/or an air quality sensor. Additionally, the one or more sensor systems 810 may utilize any suitable sensing technology, including, but not limited to, interferometric, magnetic, capacitive, ultrasonic, resistive, optical, acoustic, piezoelectric, or thermal technologies.

The I/O mechanism 812 may transmit or receive data from a user or another electronic device. The I/O mechanism 812 may include the electronic display 802, a touch sensing input surface, a crown, one or more buttons (e.g., a graphical user interface “home” button), one or more cameras (including an under-display camera), one or more microphones or speakers, one or more ports such as a microphone port, and/or a keyboard. Additionally or alternatively, the I/O mechanism 812 may transmit electronic signals via a communications interface, such as a wireless, wired, and/or optical communications interface. Examples of wireless and wired communications interfaces include, but are not limited to, cellular and Wi-Fi communications interfaces.

The foregoing description, for purposes of explanation, uses specific nomenclature to provide a thorough understanding of the described embodiments. However, it will be apparent to one skilled in the art, after reading this description, that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art, after reading this description, that many modifications and variations are possible in view of the teachings herein.

As described herein, one aspect of the present technology may be the gathering and use of data available from various sources, including biometric data (e.g., information about a person's respiration and movement). The present disclosure contemplates that, in some instances, this gathered data may include personal information data that uniquely identifies or can be used to identify, locate, or contact a specific person. Such personal information data can include, for example, biometric data and data linked thereto (e.g., demographic data, location-based data, telephone numbers, email addresses, home addresses, data or records relating to a user's health or level of fitness (e.g., vital signs measurements, medication information, exercise information), date of birth, or any other identifying or personal information).

The present disclosure recognizes that the use of such personal information data, in the present technology, can be used to the benefit of users. For example, the personal information data can be used to authenticate a user to access their device, or gather performance metrics for the user's interaction with an augmented or virtual world. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services or anytime thereafter. In another example, users can select not to provide data to targeted content delivery services. In yet another example, users can select to limit the length of time data is maintained or entirely prohibit the development of a baseline profile for the user. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other nonpersonal information available to the content delivery services, or publicly available information.

What is claimed is:

1. A head-mounted device, comprising:
a housing; and
a set of one or more self-mixing interferometry (SMI) sensors disposed in the housing and configured to:
emit electromagnetic radiation toward an anatomical structure adjacent a nasal passageway of a user; and
generate one or more SMI signals including information about movement of the anatomical structure.

2. The wearable device of claim 1, further comprising processing circuitry communicably coupled to the set of one or more SMI sensors and configured to determine respiration information about the user based on the one or more SMI signals.

3. The wearable device of claim 2, wherein the processing circuitry is further configured to detect a facial movement of the user based on the one or more SMI signals.

4. The wearable device of claim 2, wherein the respiration information comprises one or more of:
respiration rate;
respiration quality;
information about nasal congestion;
information about snoring;
airflow velocity; and
breathing volume.

5. The wearable device of claim 1, further comprising a display positioned and oriented to be over at least one eye of the user.

6. The wearable device of claim 5, further comprising processing circuitry communicably coupled to the set of one or more SMI sensors and the display, the processing circuitry configured to detect a facial movement of the user based on the one or more SMI signals.

7. The wearable device of claim 6, wherein the processing circuitry is further configured to change a user interface shown on the display in response to the detection of the facial movement of the user.

8. The wearable device of claim 1, wherein the wearable device comprises a mask configured to be worn over a nose and mouth of the user.

9. The wearable device of claim 1, wherein the wearable device comprises eyewear configured to be worn over eyes of the user.

10. A wearable device, comprising:
a housing;
a set of one or more self-mixing interferometry (SMI) sensors disposed in the housing and configured to:
emit electromagnetic radiation towards an anatomical structure a user; and
generate one or more SMI signals including information about the anatomical structure;
processing circuitry communicably coupled to the set of one or more SMI sensors and configured to:
determine if it is appropriate to emit electromagnetic radiation from the set of one or more SMI sensors;
enable the emission of electromagnetic radiation from the set of one or more SMI sensors when it is appropriate to emit electromagnetic radiation from the set of one or more SMI sensors; and
disable the emission of electromagnetic radiation from the set of one or more SMI sensors when it is not appropriate to emit electromagnetic radiation from the set of one or more SMI sensors.

11. The wearable device of claim 10, wherein the processing circuitry is configured to determine if it is appropriate to emit electromagnetic radiation from the set of one or more SMI sensors based on the one or more SMI signals.

12. The wearable device of claim 10, further comprising:
a proximity sensor disposed in the housing near the set of one or more SMI sensors and configured to generate a proximity signal indicative of whether the wearable device is proximate to the user; wherein,
the processing circuitry is communicably coupled to the proximity sensor and configured to determine if it is appropriate to emit electromagnetic radiation from the set of one or more SMI sensors based on the proximity signal.

13. The wearable device of claim 10, wherein:
the wearable device is configured to be worn on a head of the user; and
the set of one or more SMI sensors are disposed in the housing such that they are positioned near an eye of the user.

14. The wearable device of claim 10, wherein the anatomical structure is a respiratory pathway.

15. The wearable device of claim 10, wherein the set of one or more SMI sensors is disposed in a portion of the housing configured to be positioned over a portion of a nose of the user.

16. A method of operating a wearable device, comprising:
generating, from a set of one or more self-mixing interferometry (SMI) sensors of the wearable device, one or more SMI signals including information about movement of tissue near a respiratory pathway of a user; and
determining, by a processing system of the wearable device, respiration information about the user based on the one or more SMI signals.

17. The method of claim 16, wherein the respiration information comprises one or more of:
respiration rate;
respiration quality;
information about nasal congestion;
information about snoring;
airflow velocity; and
breathing volume.

18. The method of claim 16, wherein the tissue is bone.

19. The method of claim 16, wherein the tissue is soft tissue.

20. The method of claim 16, wherein the respiratory pathway is a nasal passageway.
